(19) United States
(12) Patent Application Publication
(10) Pub. No.: US 2024/0285348 A1
(43) Pub. Date: Aug. 29, 2024

(54) AUTOMATED MOVEMENT OF OPTICAL LOCALIZER FOR OPTIMAL LINE OF SIGHT WITH OPTICAL TRACKERS

(71) Applicant: Mazor Robotics Ltd., Caesarea (IL)

(72) Inventors: Adi SANDELSON, Givatayim (IL); Elad RATZABI, Bait Herut (IL)

(21) Appl. No.: 18/175,640

(22) Filed: Feb. 28, 2023

(51) Int. Cl. A61B 34/20

(52) U.S. Cl. CPC A61B 34/20; A61B 2034/2055; A61B 2034/2065; A61B 90/37

(57) ABSTRACT

A surgical navigation system including an optical tracking device, an optical localizer configured to optically track the optical tracking device, a mount assembly supporting the optical localizer, and a processor. The processor is configured to transmit commands to the mount assembly for actuating the mount assembly to position the optical localizer where there is line of sight between the optical localizer and the optical tracking device.

### FIELD

The present disclosure relates to optical navigation of a subject, and particularly to systems and methods for automated movement of an optical localizer for optimal line of sight with optical trackers.

### BACKGROUND

This section provides background information related to the present disclosure, which is not necessarily prior art.

An instrument can be navigated relative to a subject for performing various procedures. For example, the subject can include a patient on which a surgical procedure is being performed. During a surgical procedure, an instrument can be tracked in an object or subject space. In various embodiments the subject space can be a patient space defined by a patient. The location of the instrument that is tracked can be displayed on a display device relative to an image of the patient.

The position of the patient can be determined with a tracking system. Generally, a patient is registered to the image, via tracking an instrument relative to the patient to generate a translation map between the subject or object space (e.g. patient space) and the image space. This often requires time during a surgical procedure for a user, such as a surgeon, to identify one or more points in the subject space and correlating, often identical points, in the image space.

After registration, the position of the instrument can be appropriately displayed on the display device while tracking the instrument. The position of the instrument relative to the subject can be displayed as a graphical representation, sometimes referred to as an icon on the display device.

During a navigated procedure or a robotic-navigated procedure, the support staff may need to manually adjust position of an optical localizer multiple times to be able to capture the reference frame and the navigated tools in the work volume of the optical localizer. The present disclosure advantageously reduces or eliminates any need for manual adjustment of the optical localizer.

### SUMMARY

This section provides a general summary of the disclosure, and is not a comprehensive disclosure of its full scope or all of its features.

The present disclosure includes a system and method to maintain a line of sight of a localizer and a tracking device. The localizer and/or the tracking device may move during a procedure. A line of sight between the two may be lost. The system and method disclosed allows to a reacquisition or reestablishment of the line of sight.

According to various embodiments, a surgical navigation system that may include a tracking device and a localizer configured to track the tracking device is disclosed. The system may further include a mount assembly supporting the localizer. Also, a processor may execute instructions to transmit commands to the mount assembly for actuating the mount assembly to position the localizer where there is line of sight between the localizer and the tracking device. The tracking device and the localizer may be optical embodiments.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of select embodiments and not all possible implementations, and are not intended to limit the scope of the present disclosure.

FIG. 1 is diagrammatic view illustrating an overview of a robotic system and a navigation system, according to various embodiments;

FIG. 2 is a detailed environmental view of a robotic system and a tracking system, according to various embodiments;

FIG. 3 is a detailed view of a robotic system with a snapshot tracking device, according to various embodiments;

FIG. 4 illustrates an exemplary robotic arm and optical localizer supported at a first position by a mount assembly-optical line of sight between the robotic arm and the optical localizer is blocked by an obstruction;

FIG. 5 illustrates the robotic arm and optical localizer supported at a second position by the mount assembly-optical line of sight between the robotic arm and the optical localizer is blocked by the obstruction;

FIG. 6 illustrates the robotic arm and optical localizer supported at a third position by the mount assembly-optical line of sight between the robotic arm and the optical localizer is not blocked by the obstruction;

FIG. 7 is a top view of the optical localizer illustrating exemplary range of motion;

FIG. 8 illustrates the optical localizer and two exemplary optical tracking devices-optical line of sight between the optical localizer and the exemplary optical tracking devices is blocked by obstructions;

FIG. 9 illustrates an exemplary three-dimensional coordinate system through which the mount assembly is configured to maneuver the optical localizer to establish line of sight with an optical tracker; and

FIG. 10 illustrates an exemplary method in accordance with the present disclosure for maintaining line of sight between an optical localizer and an optical tracking device.

Corresponding reference numerals indicate corresponding parts throughout the several views of the drawings.

### DETAILED DESCRIPTION

Example embodiments will now be described more fully with reference to the accompanying drawings.

The subject disclosure is directed to an exemplary embodiment of a surgical procedure on a subject, such as a human patient. It is understood, however, that the system and methods described herein are merely exemplary and not intended to limit the scope of the claims included herein.

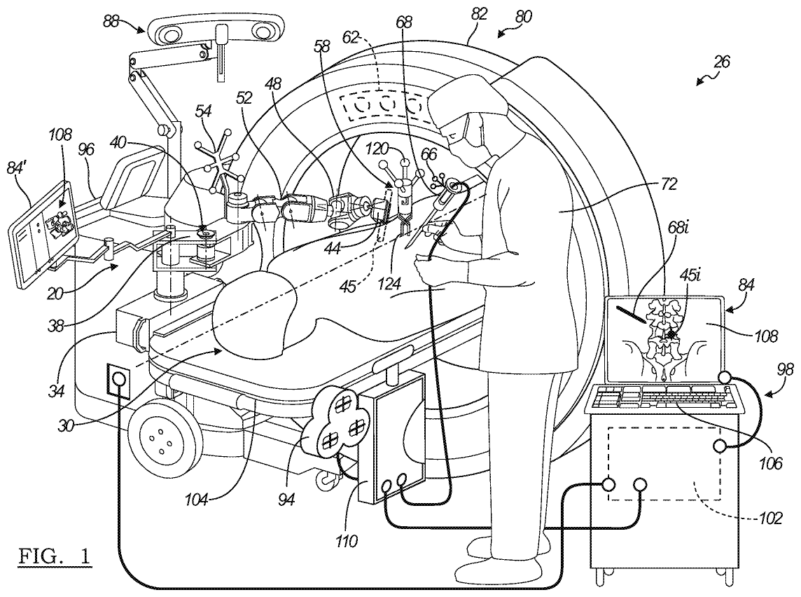

FIG. 1 is a diagrammatic view illustrating an overview of a procedure room or arena. In various embodiments, the procedure room may include a surgical suite in which may be placed a robotic system 20 and a navigation system 26 that can be used for various procedures. The robotic system 20 may include a Mazor X™ robotic guidance system, sold by Medtronic, Inc. The robotic system 20 may be used to assist in guiding selected instrument, such as drills, screws, etc. relative to a subject 30. The robotic system 20 may include a mount 34 that fixes a portion, such as a robotic base 38, relative to the subject 30. The robotic system 20 may include one or more arms 40 that are moveable or pivotable relative to the subject 30, such as including an end effector 44. The end effector may be any appropriate portion, such as a tube, guide, or passage member. The end effector 44 may be moved relative to the base 38 with one or more motors. The position of the end effector 44 may be known or determined relative to the base 38 with one or more encoders at one or more joints, such as a wrist joint 48 and/or an elbow joint 52 of the robotic system 20.

The navigation system 26 can be used to track the location of one or more tracking devices, tracking devices may include a robot tracking device 54, a subject tracking device 58, an imaging system tracking device 62, and/or an tool tracking device 66. A tool 68 may be any appropriate tool such as a drill, forceps, or other tool operated by a user 72. The tool 68 may also include an implant, such as a spinal implant or orthopedic implant. It should further be noted that the navigation system 26 may be used to navigate any type of instrument, implant, or delivery system, including: guide wires, arthroscopic systems, orthopedic implants, spinal implants, deep brain stimulation (DBS) probes, etc. Moreover, the instruments may be used to navigate or map any region of the body. The navigation system 26 and the various instruments may be used in any appropriate procedure, such as one that is generally minimally invasive or an open procedure.

An imaging device 80 may be used to acquire pre-, intra-, or post-operative or real-time image data of a subject, such as the subject 30. It will be understood, however, that any appropriate subject can be imaged and any appropriate procedure may be performed relative to the subject. In the example shown, the imaging device 80 comprises an O-arm® imaging device sold by Medtronic Navigation, Inc. having a place of business in Louisville, Colorado, USA. The imaging device 80 may have a generally annular gantry housing 82 in which an image capturing portion is moveably placed. The image capturing portion may include an x-ray source or emission portion and an x-ray receiving or image receiving portion located generally or as practically possible 180 degrees from each other and mounted on a rotor relative to a track or rail. The image capturing portion can be operable to rotate 360 degrees during image acquisition. The image capturing portion may rotate around a central point or axis, allowing image data of the subject 80 to be acquired from multiple directions or in multiple planes. The imaging device 80 can include those disclosed in U.S. Pat. Nos. 7,188,998; 7,108,421; 7,106,825; 7,001,045; and 6,940,941; all of which are incorporated herein by reference, or any appropriate portions thereof. In one example, the imaging device 80 can utilize flat plate technology having a 1,720 by 1,024 pixel viewing area.

The position of the imaging device 80, and/or portions therein such as the image capturing portion, can be precisely known relative to any other portion of the imaging device 80. The imaging device 80, according to various embodiments, can know and recall precise coordinates relative to a fixed or selected coordinate system. This can allow the imaging system 80 to know its position relative to the patient 30 or other references. In addition, the precise knowledge of the position of the image capturing portion can be used in conjunction with a tracking system to determine the position of the image capturing portion and the image data relative to the tracked subject, such as the patient 30.

The imaging device 80 can also be tracked with a tracking device 62. The image data defining an image space acquired of the patient 30 can, according to various embodiments, be inherently or automatically registered relative to an object space. The object space can be the space defined by a patient 30 in the navigation system 26. The automatic registration can be achieved by including the tracking device 62 on the imaging device 80 and/or the determinable precise location of the image capturing portion. According to various embodiments, as discussed herein, imageable portions, virtual fiducial points and other features can also be used to allow for registration, automatic or otherwise. It will be understood, however, that image data can be acquired of any subject which will define subject space. Patient space is an exemplary subject space. Registration allows for a translation between patient space and image space.

The patient 30 can also be tracked as the patient moves with a patient tracking device, DRF, or tracker 58. Alternatively, or in addition thereto, the patient 30 may be fixed within navigation space defined by the navigation system 26 to allow for registration. As discussed further herein, registration of the image space to the patient space or subject space allows for navigation of the instrument 68 with the image data. When navigating the instrument 68, a position of the instrument 68 can be illustrated relative to image data acquired of the patient 30 on a display device 84. Various tracking systems, such as one including an optical localizer 88 or an electromagnetic (EM) localizer 94 can be used to track the instrument 68.

More than one tracking system can be used to track the instrument 68 in the navigation system 26. According to various embodiments, these can include an electromagnetic tracking (EM) system having the EM localizer 94 and/or an optical tracking system having the optical localizer 88. Either or both of the tracking systems can be used to track selected tracking devices, as discussed herein. It will be understood, unless discussed otherwise, that a tracking device can be a portion trackable with a selected tracking system. A tracking device need not refer to the entire member or structure to which the tracking device is affixed or associated.

It is further appreciated that the imaging device 80 may be an imaging device other than the O-arm® imaging device and may include in addition or alternatively a fluoroscopic C-arm. Other exemplary imaging devices may include fluoroscopes such as bi-plane fluoroscopic systems, ceiling mounted fluoroscopic systems, cath-lab fluoroscopic systems, fixed C-arm fluoroscopic systems, isocentric C-arm fluoroscopic systems, 3D fluoroscopic systems, etc. Other appropriate imaging devices can also include MRI, CT, ultrasound, etc.

In various embodiments, an imaging device controller 96 may control the imaging device 80 and can receive the image data generated at the image capturing portion and store the images for later use. The controller 96 can also control the rotation of the image capturing portion of the imaging device 80. It will be understood that the controller 96 need not be integral with the gantry housing 82, but may be separate therefrom. For example, the controller may be a portions of the navigation system 26 that may include a processing and/or control system 98 including a processing unit or processing portion 102. The controller 96, however, may be integral with the gantry 82 and may include a second and separate processor, such as that in a portable computer.

The patient 30 can be fixed onto an operating table 104. According to one example, the table 104 can be an Axis Jackson® operating table sold by OSI, a subsidiary of Mizuho Ikakogyo Co., Ltd., having a place of business in Tokyo, Japan or Mizuho Orthopedic Systems, Inc. having a place of business in California, USA. Patient positioning devices can be used with the table, and include a Mayfield® clamp or those set forth in commonly assigned U.S. patent application Ser. No. 10/405,068 entitled “An Integrated Electromagnetic Navigation And Patient Positioning Device”, filed Apr. 1, 2003 which is hereby incorporated by reference.

The position of the patient 30 relative to the imaging device 80 can be determined by the navigation system 26. The tracking device 62 can be used to track and locate at least a portion of the imaging device 80, for example the gantry or housing 82. The patient 30 can be tracked with the dynamic reference frame 58, as discussed further herein. Accordingly, the position of the patient 30 relative to the imaging device 80 can be determined. Further, the location of the imaging portion can be determined relative to the housing 82 due to its precise position on the rail within the housing 82, substantially inflexible rotor, etc. The imaging device 80 can include an accuracy of within 10 microns, for example, if the imaging device 80 is an O-Arm® imaging device sold by Medtronic Navigation, Inc. having a place of business in Louisville, Colorado. Precise positioning of the imaging portion is further described in U.S. Pat. Nos. 7,188,998; 7,108,421; 7,106,825; 7,001,045; and 6,940,941; all of which are incorporated herein by reference,

According to various embodiments, the imaging device 80 can generate and/or emit x-rays from the x-ray source that propagate through the patient 30 and are received by the x-ray imaging receiving portion. The image capturing portion generates image data representing the intensities of the received x-rays. Typically, the image capturing portion can include an image intensifier that first converts the x-rays to visible light and a camera (e.g. a charge couple device) that converts the visible light into digital image data. The image capturing portion may also be a digital device that converts x-rays directly to digital image data for forming images, thus potentially avoiding distortion introduced by first converting to visible light.

Two dimensional and/or three dimensional fluoroscopic image data that may be taken by the imaging device 80 can be captured and stored in the imaging device controller 96. Multiple image data taken by the imaging device 80 may also be captured and assembled to provide a larger view or image of a whole region of a patient 30, as opposed to being directed to only a portion of a region of the patient 30. For example, multiple image data of the patient's 30 spine may be appended together to provide a full view or complete set of image data of the spine.

The image data can then be forwarded from the image device controller 96 to the navigation computer and/or processor system 102 that can be a part of a controller or work station 98 having the display 84 and a user interface 106. It will also be understood that the image data is not necessarily first retained in the controller 96, but may also be directly transmitted to the work station 98. The work station 98 can provide facilities for displaying the image data as an image 108 on the display 84, saving, digitally manipulating, or printing a hard copy image of the received image data. The user interface 106, which may be a keyboard, mouse, touch pen, touch screen or other suitable device, allows the user 72 to provide inputs to control the imaging device 80, via the image device controller 96, or adjust the display settings of the display 84. The work station 98 may also direct the image device controller 96 to adjust the image capturing portion of the imaging device 80 to obtain various two-dimensional images along different planes in order to generate representative two-dimensional and three-dimensional image data.

With continuing reference to FIG. 1, the navigation system 26 can further include the tracking system including either or both of the electromagnetic (EM) localizer 94 and/or the optical localizer 88. The tracking systems may include a controller and interface portion 110. The controller 110 can be connected to the processor portion 102, which can include a processor included within a computer. The EM tracking system may include the STEALTHSTATION® AXIEM™ Navigation System, sold by Medtronic Navigation, Inc. having a place of business in Louisville, Colorado; or can be the EM tracking system described in U.S. patent application Ser. No. 10/941,782, filed Sep. 15, 2004, and entitled “METHOD AND APPARATUS FOR SURGICAL NAVIGATION”; U.S. Pat. No. 5,913,820, entitled “Position Location System,” issued Jun. 22, 1999; and U.S. Pat. No. 5,592,939, entitled “Method and System for Navigating a Catheter Probe,” issued Jan. 14, 1997; all of which are herein incorporated by reference. It will be understood that the navigation system 26 may also be or include any appropriate tracking system, including a STEALTHSTATION® TREON® or S7™ tracking systems having an optical localizer, that may be used as the optical localizer 88, and sold by Medtronic Navigation, Inc. of Louisville, Colorado. Other tracking systems include an acoustic, radiation, radar, etc. The tracking systems can be used according to generally known or described techniques in the above incorporated references. Details will not be included herein except when to clarify selected operation of the subject disclosure.

Wired or physical connections can interconnect the tracking systems, imaging device 80, etc. Alternatively, various portions, such as the instrument 68 may employ a wireless communications channel, such as that disclosed in U.S. Pat. No. 6,474,341, entitled “Surgical Communication Power System,” issued Nov. 5, 2002, herein incorporated by reference, as opposed to being coupled directly to the controller 110. Also, the tracking devices 62, 66, 54 can generate a field and/or signal that is sensed by the localizer(s) 88, 94.

Various portions of the navigation system 26, such as the instrument 68, and others as will be described in detail below, can be equipped with at least one, and generally multiple, of the tracking devices 66. The instrument can also include more than one type or modality of tracking device 66, such as an EM tracking device and/or an optical tracking device. The instrument 68 can include a graspable or manipulable portion at a proximal end and the tracking devices may be fixed near the manipulable portion of the instrument 68.

Additional representative or alternative localization and tracking system is set forth in U.S. Pat. No. 5,983,126, entitled “Catheter Location System and Method,” issued Nov. 9, 1999, which is hereby incorporated by reference. The navigation system 26 may be a hybrid system that includes components from various tracking systems.

According to various embodiments, the navigation system 26 can be used to track the instrument 68 relative to the patient 30. The instrument 68 can be tracked with the tracking system, as discussed above. Image data of the patient 30, or an appropriate subject, can be used to assist the user 72 in guiding the instrument 68. The image data, however, is registered to the patient 30. The image data defines an image space that is registered to the patient space defined by the patient 30.

Generally, registration allows a translation map to be generated of the physical location of the instrument 68 relative to the image space of the image data. The translation map allows the tracked position of the instrument 68 to be displayed on the display device 84 relative to the image data 108. A graphical representation 68i, also referred to as an icon, can be used to illustrate the location of the instrument 68 relative to the image data 108.

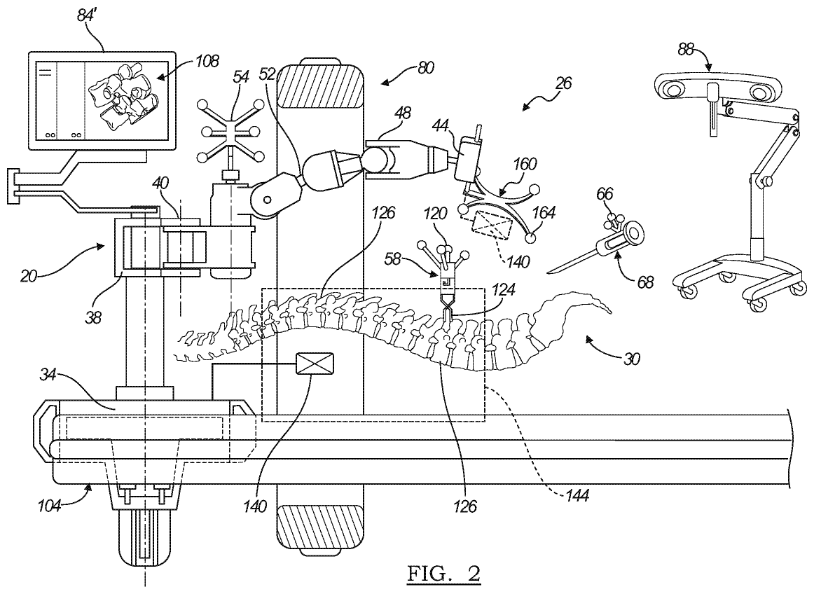

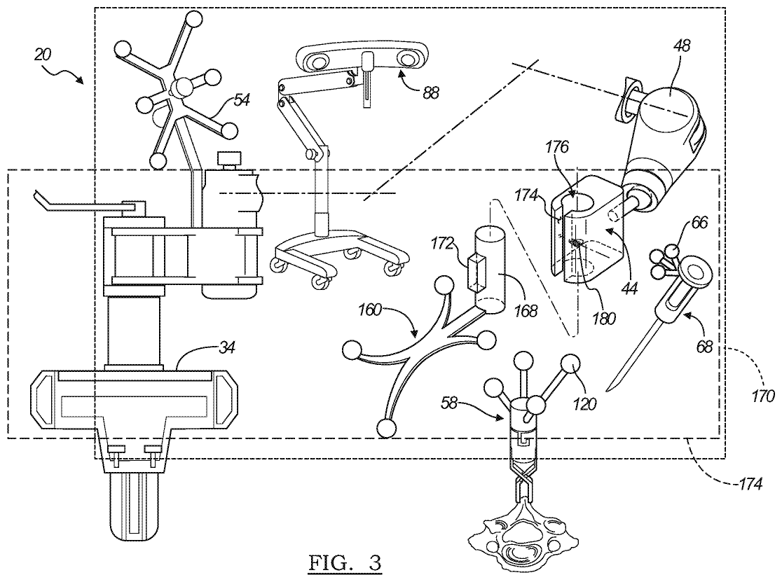

With continuing reference to FIG. 1 and additional reference to FIG. 2 and FIG. 3, a subject registration system or method can use the tracking device 58. The tracking device 58 may include portions or members 120 that may be trackable, but may also act as or be operable as a fiducial assembly. The fiducial assembly 120 can include a clamp or other fixation portion 124 and the imageable fiducial body 120. It is understood, however, that the members 120 may be separate from the tracking device 58. The fixation portion 124 can be provided to fix any appropriate portion, such as a portion of the anatomy. As illustrated in FIGS. 1 and 2, the fiducial assembly 120 can be interconnected with a portion of a spine 126 such as a spinous process 130.

The fixation portion 124 can be interconnected with a spinous process 130 in any appropriate manner. For example, a pin or a screw can be driven into the spinous process 130. Alternatively, or in addition thereto, a clamp portion 124 can be provided to interconnect the spinous process 130. The fiducial portions 120 may be imaged with the imaging device 80. It is understood, however, that various portions of the subject (such as a spinous process) may also be used as a fiducial portion.

In various embodiments, when the fiducial portions 120 are imaged with the imaging device 80, image data is generated that includes or identifies the fiducial portions 120. The fiducial portions 120 can be identified in image data automatically (e.g. with a processor executing a program), manually (e.g. by selection an identification by the user 72), or combinations thereof (e.g. by selection an identification by the user 72 of a seed point and segmentation by a processor executing a program). Methods of automatic imageable portion identification include those disclosed in U.S. Pat. No. 8,150,494 issued on Apr. 3, 2012, incorporated herein by reference. Manual identification can include selecting an element (e.g. pixel) or region in the image data wherein the imageable portion has been imaged. Regardless, the fiducial portions 120 identified in the image data can be used as fiducial points or positions that can be used to register the image data or the image space of the image data with patient space.

In various embodiments, to register an image space or coordinate system to another space or coordinate system, such as a navigation space, the fiducial portions 120 that are identified in the image 108 may then be identified in the subject space defined by the subject 30, in an appropriate manner. For example, the user 72 may move the instrument 68 relative to the subject 30 to touch the fiducial portions 120, if the fiducial portions are attached to the subject 30 in the same position during the acquisition of the image data to generate the image 108. It is understood that the fiducial portions 120, as discussed above in various embodiments, may be attached to the subject 30 and/or may include anatomical portions of the subject 30. Additionally, a tracking device may be incorporated into the fiducial portions 120 and they may be maintained with the subject 30 after the image is acquired. In this case, the registration or the identification of the fiducial portions 120 in a subject space may be made. Nevertheless, according to various embodiments, the user 72 may move the instrument 68 to touch the fiducial portions 120. The tracking system, such as with the optical localizer 88, may track the position of the instrument 68 due to the tracking device 66 attached thereto. This allows the user 72 to identify in the navigation space the locations of the fiducial portions 120 that are identified in the image 108. After identifying the positions of the fiducial portions 120 in the navigation space, which may include a subject space, the translation map may be made between the subject space defined by the subject 30 in a navigation space and the image space defined by the image 108. Accordingly, identical or known locations allow for registration as discussed further herein.

During registration, a translation map is determined between the image data coordinate system of the image data such as the image 108 and the patient space defined by the patient 30. Once the registration occurs, the instrument 68 can be tracked with the tracking system that is registered to the image data to allow an identification and illustration of a position of the tracked instrument 68 as an icon superimposed on the image data. Registration of the image 108 (or any selected image data) to the subject 30 may occur at any appropriate time.

After the registration of the image space to the patient space, the instrument 68 can be tracked relative to the image 108. As illustrated in FIG. 1, the icon 68i representing a position (which may include a 6 degree of freedom position (including 3D location and orientation)) of the instrument 68 can be displayed relative to the image 108 on the display 84. Due to the registration of the image space to the patient space, the position of the icon 68i relative to the image 108 can substantially identify or mimic the location of the instrument 68 relative to the patient 30 in the patient space. As discussed above, this can allow a navigated procedure to occur.

The robotic system 20 having the robotic system coordinate system may be registered to the navigation space coordinate system, as discussed herein, due to the reference tracking device 54 (e.g. if fixed to a known position on or relative to the robotic system 20) and/or due to the tracking of the snapshot tracking device 160. Due to or during registration, a translation map is determined between the robotic system coordinate system of the robotic system and the navigation space coordinate system patient space defined by the patient 30. The snapshot tracking device 160 may include one or more trackable portions 164 that may be tracked with the localizer 88 or any appropriate localizer (e.g. optical, EM, radar). It is understood, however, that any appropriate tracking system may be used to track the snapshot tracking device 160. A fixed reference tracking device may also be positioned within the navigation space. The fixed navigation tracker may include the patient tracker 58 which may be connected to the patient 30 and/or the robot tracker 54 that may be fixed to the mount 34 of the robotic system 20. The reference tracker, therefore, may be any appropriate tracker that is positioned relative to the snapshot tracker 160 that is within the navigation coordinate space during the registration period. For the discussion herein the robot tracker 54 will be referred to however, the patient tracker 58 may also be used as the reference tracker. Further, reference tracker may be positioned within the coordinate system at any position relative to the snapshot tracker 160 as long as the snapshot tracker 160 may be tracked relative to the reference tracker.

In various embodiments, the snapshot tracker 160 may be positioned at a known position relative to the end effector 44. For example, the snapshot tracker 160, as illustrated in FIG. 3, which includes the trackable portions 164, extends from a rod or connection member 168. The connection member 168 may include a keyed portion, such as a projection 172 that may engage a slot 174 of the end effector 44. The end effector 44 may form or define a cannula or passage 176 that may engage the connector 168. The connector 168 may be positioned within the passage 176 of the end effector 44. The connector 168 may then be fixed to the end effector 44, such as with a fixation member including a set screw or clamping of the end effector 44, such as with a set screw or clamping member 180. The projection 172 may engage within the slot 174 to fix the snapshot tracker 160 rotationally relative to the end effector 44. The connector 168 positioned within the passage 176 and locked in place with the set screw 180 may then rigidly fix the snapshot tracking device 160 relative to the end effector 44. Thus, the position of the snapshot tracker 160 relative to the end effector 44 may be fixed.

The localizer 88 may then view or determine a position of the snapshot tracking device 160 relative to the reference tracking device 54 and or the reference tracking device 58. As the localizer 88 defines or may be used to define the navigation space, determining or tracking a position of the snapshot localizer 160 relative to the reference frame 54 may be used to determine a relationship between a position within the navigation space and the robotic space of the end effector 44.

With continuing reference to FIG. 3, therefore, the navigation space defined by the localizer 88 may include the full navigation space 170 which may include portions relative to the subject, such as the subject tracker 58 and other portions that may be moved therein, such as the instrument 68. The robotic registration space may be smaller and may include a robotic registration space 174 that may include the reference frame 54 and the snapshot tracker 160. As discussed above, however, the robot registration navigation space may include the snapshot tracker 160 and the patient tracker 58 for registration. Accordingly, the exemplary registration navigation space 174 is merely for the current discussion. Both the robotic reference tracker 54 and the patient tracker 58 need not be used simultaneously. This is particularly true when the patient 30 is fixed in space, such as fixed relative to the robotic system 20.

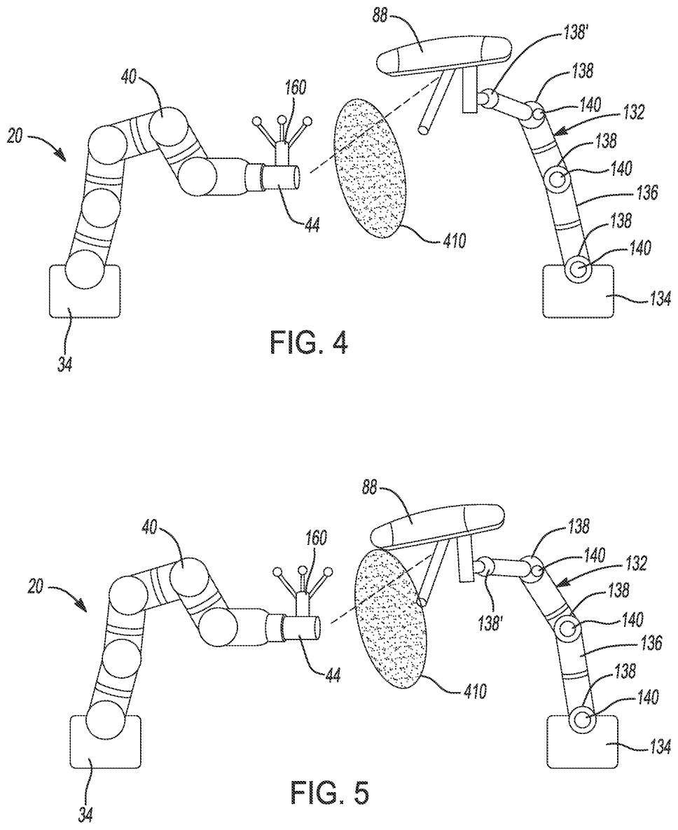

With additional reference to FIGS. 4-8, the optical localizer 88 is supported by a mount assembly 132, which generally includes a base 134 and an arm 136. The arm 136 is mounted to, and extends from, the base 134. The arm 136 includes a plurality of joints 138. The joints 138 may be ball-in-socket joints, for example, or any other suitable joints. The arm 136 and the joints 138 are configured such that at the joints 138 the arm 136 can bend, rotate, extend, contract, or otherwise actuate to position the optical localizer 88 at any suitable location. The joints 138 thus provide the arm 136 with at least six degrees of freedom, for example.

One or more of the joints 138 includes a device configured to mechanically maneuver the arm 136. For example, one or more of the joints 138 may include a servomotor 140, or any other suitable device configured to bend, rotate, extend, contract, or otherwise actuate the arm 136 at the joints 138. With particular reference to FIG. 7, the joint 138′ closest to the optical localizer 88 includes a servomotor 140′ configured to move the optical localizer 88 left and right as illustrated. The servomotor 140′ is further configured to move the optical localizer 88 up and down.

Actuation of the mount assembly 132 to position the optical localizer 88 is controlled by the processor 102 of the work station 98, or any other suitable processing device. For the optical localizer 88 to optically track an optical tracking device, optical line of sight must be maintained between the optical localizer 88 and the tracking device. The optical localizer 88 is configured to track any suitable tracking devices, such as, but not limited to, the optical tracking devices 160, 58, 66, 410A, 410B. When the optical line of sight is blocked, tracking cannot occur. For example, and as illustrated in FIG. 4, tracking cannot occur when an obstruction 410 is between the optical localizer 88 and the snapshot optical tracking device 160 of the robotic system 20. The obstruction 410 may be a surgeon, nurse, or any other person in the procedure room. The obstruction may also be, for example, equipment in the procedure room.

With respect to the robotic system 20 of FIGS. 4-6, the processor 102 of the workstation 98, or any other suitable processor, is in communication with the robotic system 20 to actuate the robotic arm 40. Communication between the robotic arm 40 and the processor 102 is such that the processor 102 knows the orientation and position of the robotic arm 40 within the three-dimensional coordinate system of the arm 40. The position of the snapshot tracking device 160 and the end effector 44 relative to the robotic arm 40 is input to the processor 102. Thus, the processor 102 also knows the positions of the snapshot tracking device 160 and the end effector 44 within the three-dimensional coordinate system of the arm 40.

When the snapshot optical tracking device 160 is visible to the optical localizer 88, the location of the optical tracking device 160 (as well as the end effector 44 and the robotic arm 40 generally) within a coordinate system of the optical localizer 88 is known and input to the processor 102. That is the processor 102 is configured to execute instructions to know the location (including spatial coordinates and orientation) of the optical tracking device 160 when it is visible to the optical localizer 80. The relative location of the portions attached or fixed to the optical tracking device 160 may also be known or recalled from a selected memory system. The processor 102 also knows the location of the snapshot tracking device 160 within the coordinate system of the robotic arm 40 at all times. Based on known locations of the snapshot optical tracking device 160 in both the coordinate system of the robotic arm 40 and the coordinate system of the optical localizer 88, the processor 102 is configured to execute instructions to determine a position the optical localizer 88 in the coordinate system of the robotic arm 40. This is, at least in part, due to the registration or correlation of the optical localizer 88 coordinate system and the coordinate system of the robotic arm 40 at least as discussed above.

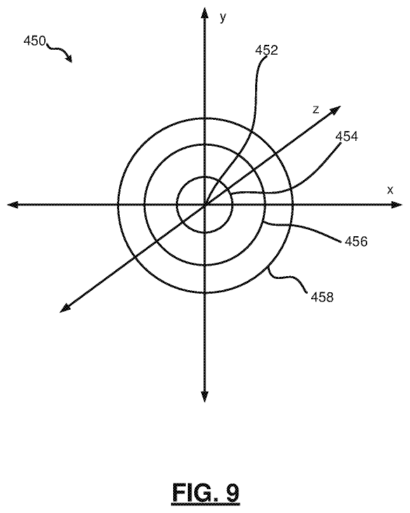

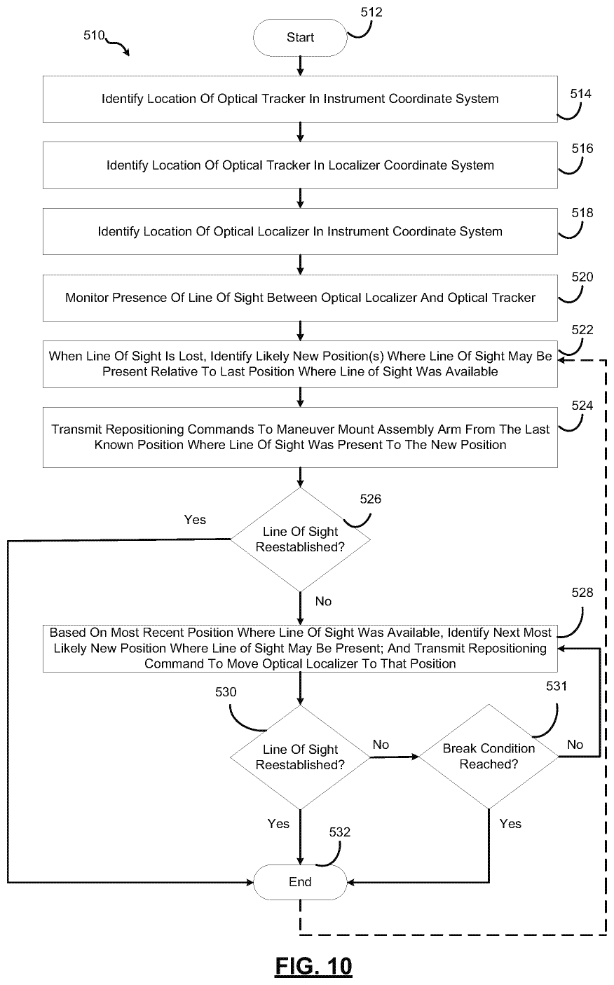

In a situation where line of sight between the optical localizer 88 and the optical tracking device 160 is lost, such as due to presence of the obstruction 410 therebetween, the processor 102 recalls the last known position of the optical localizer 88 in the coordinate system of the robotic arm 40 when line of sight was available. This last known position is illustrated graphically on the three-dimensional axis 450 of FIG. 9 at reference numeral 452. An attempt to reestablish line of sight may then occur. Such a process may be referred to as a search or search pattern, as discussed herein.

The processor 102, therefore, may transmit instructions to the mount assembly 132 for actuating the servomotors 140, 140′ to move the optical localizer 88 a predetermined distance from the position 452, such as 150 mm for example, out to a location along a first coordinate ring 456 of FIG. 9. The ring 456 may be 150 mm from the position 452, or any other suitable distance. The processor 102 then sends instructions to the mount assembly 132 for maneuvering the optical localizer 88 along the coordinate ring 456. To do this, the processor 102 instructs the mount assembly 132 to maneuver the optical localizer 88 throughout the six-degrees of freedom (e.g., up, down, left, right, in, out, etc.) and/or in a coordinated movement in the six degrees of freedom. For example, moving the optical localizer 88 generally in the circle of the coordinate ring 456. This coordinated movement allows a field of view of the optical localizer 88 to move along the path defined by the coordinate ring 456. This may be moving the localizer 80 in a circle defined in the 3D coordinate space the selected distance from the point 452. The processor 102 may also instruct the mount assembly 132 to rotate the optical localizer 88, as illustrated in FIG. 7.

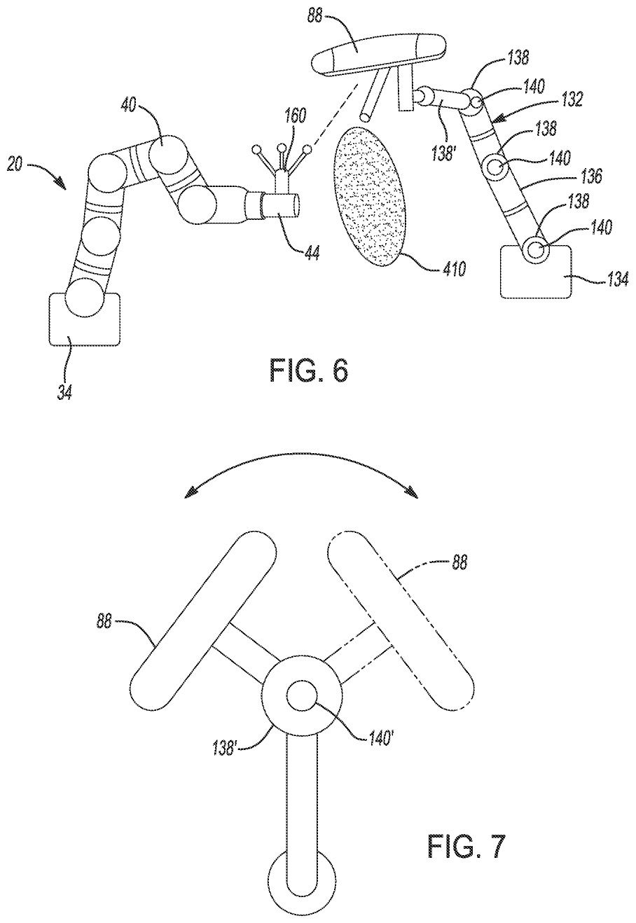

The processor 102 maneuvers the optical localizer 88 along the first coordinate ring 456 in an attempt to reestablish line of sight. If line of sight is not reestablished, as is the case in FIG. 5, then the processor 102 moves the optical localizer 88 further from the last position 452 where optical line of sight was present, such as to a second coordinate ring 458. The second coordinate ring 458 may be 150 mm from the first coordinate ring 456, or any other suitable distance. The processor 102 maneuvers the optical localizer 88 along the second coordinate ring 458 in an attempt to reestablish line of sight, in a manner similar to that noted above. If line of sight is not reestablished, then the processor 102 moves the optical localizer 88 out to additional coordinate rings until optical line of sight is ultimately reestablished, as illustrated in FIG. 6.

Although the above description indicates that the optical localizer 88 is moved about “coordinate rings” 456, 458, the optical localizer 88 may be moved not just along “rings,” but in any suitable manner during the “search” to reestablish line of sight. For example, the optical localizer 88 may be moved along a line/vector along which the optical tracking device 160 was last known to be present and/or moving. Thus, given the known last location and known last motion (e.g., speed and direction) a search pattern may be determined to be a in a selected direction along a selected line.

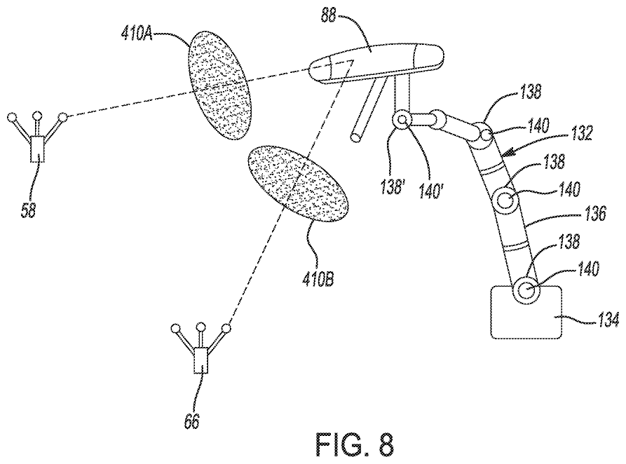

FIG. 8 illustrates an application where the optical localizer 88 is used to track the subject tracking device 58 and the tool tracking device 66, or any other suitable non-robotic tracking device. In this application, positions of the tracking devices 58, 66 are known in the coordinate system of the optical localizer 88 as a result of prior registration. The processor 102 is configured to operate the optical localizer 88 to “lock on” to the tracking devices 58, 68, and/or any other suitable targets, and identify one or more optimal positions where the optical localizer 88 can best see the tracking devices 58, 68 and/or other targets. The processor 102 is further configured to translate the optimal position(s) of the optical localizer 88 into movement of the optical localizer 88 to the optimal position(s) using kinematic calculations that may be done without any involvement from users. In case of loss of line of sight, such as due to the presence of the obstruction 410A and/or the obstruction 410B, the processor 102 maneuvers the optical localizer 88 throughout the three-dimensional coordinate plane 450 in the same manner described above.

FIG. 10 illustrates an exemplary method 510 for maintaining line of sight between the optical localizer 88 and an optical tracking device, such as any one or more of the optical tracking devices 160, 58, 66, 410A, 410B, for example. The method 510 is described with respect to the optical tracking device 160 for exemplary purposes only. The method 510 starts at block 512, where the method 510 is initiated by the processor 102, or any other suitable processor. From block 512, the method 510 proceeds to block 514.

At block 514, the processor 102 identifies location of the optical tracker 160 in an instrument coordinate system. From block 514, the method 510 proceeds to block 516. At block 516, the processor 102 identifies location of the localizer in a localizer coordinate system. From block 516, the method 510 proceeds to block 518. At block 518, the processor 102 identifies location of the optical localizer 88 in the instrument coordinate system. From block 518, the method 510 proceeds to block 520.

At block 520, the processor 102 monitors whether there is line of sight between the optical localizer 88 and the optical tracker 160. When line of sight is lost, the processor 102 is configured to transmit repositioning commands to the mount assembly 132 to maneuver the arm 136 by actuating the servomotors 140, which repositions the optical localizer 88 in an attempt to regain line of sight. Specifically, at block 522 a search or search pattern may be initiated. At block 522 the processor 102 identifies a likely new position, positions, or range of positions where line of sight is most likely to be present. For example and as explained above, the processor 102 may recall from memory, or remotely access, the three-dimensional coordinate space 450 including a series of coordinate rings 456, 458, etc. The coordinate rings 456, 458, etc. may be arranged any suitable distance from the last position where line of sight was present at 452. For example, the coordinate rings 456, 458 may be arranged in 150 mm intervals. The coordinate rings 456, 458, etc. may be configured as rings, or have any other suitable shape. For example, the coordinate rings 456, 458 may be configured with a customized shape corresponding to the procedure room and items therein.

The method 510, including instructions that are being executed by a processor, may evaluate various considerations determining or evaluating likely new positions. For example, considerations as to a design of items that are moving may be made. This may include size, geometry, freedom of movement, etc. In various embodiments, for example, the movable portions and/or items to be moved around may include handheld trackers and/or trackers on the robot to predict where they moved. Regarding the robot all the information is available so the arm can move in relation to the known movement of the robot tracker.

At block 524, the processor 102 transmits repositioning commands to the mount assembly 132 to actuate the servomotors 140, 140′ in a manner to move the optical localizer 88 throughout a path in the procedure room corresponding to the coordinate ring 456. From block 524, the method 510 proceeds to block 526. If at block 526 the processor 102 determines that line of sight between the optical localizer 88 and the optical tracker 160 has been reestablished, then the processor 102 proceeds to block 532 where the method 510 ends. Alternatively, the method 510 may return to block 522 where the processor 102 continues to monitor for the presence of line of sight between the optical localizer 88 and the optical tracker 160.

If at block 526 line of sight has not been reestablished, then the method 510 proceeds to block 528. At block 528, the processor 102 identifies the next most likely position, positions, or range of positions where optical line of sight is likely to be present. In the example of FIG. 9, the next most likely area where line of sight is likely to be reestablished is along coordinate ring 458. The controller thus transmits instructions to the mount assembly 132 for operating the servomotors 140 to maneuver the optical localizer 88 along the coordinate ring 458 in an attempt to reestablish line of sight. From block 528, the method 510 proceeds to block 530. At block 530, the processor 102 determines whether line of sight has been reestablished.

At block 528, according to various embodiments, various features or analysis may occur to determine a likely position. For example, as the position of the robotic system is known and is correlated or registered to the navigation space, the processor 20 may know or determine the location of the tracking device 160 and determine a position to which the localizer 80 may be moved to view the tacking device 160 and/or attempt to view the tracking device 160. In addition or alternatively, the likely position may be based on a recent location, direction, and speed of movement and elapsed time since last being able to view the tracking device. The localizer 80 may be moved to a possible or likely new location of the tracing device 160 based on this information. All of this analysis may be carried out by the processor 20 by executing selected instructions. Further, the process may be iterative and/or repetitive until the tracking device 160 is within the field of view of the localizer 80, as discussed below.

If line of sight has not been reestablished, as determined in block 530, then the method 510 returns to block 528, where the processor 102 repositions the optical localizer 88 to the next most likely position, positions, or area where line of sight was established. For example, the processor 102 will instruct the mount assembly 132 to move the optical localizer along another coordinate ring outside of the coordinate ring 458. This process of repositioning the optical localizer 88 continues until line of sight is reestablished between the optical localizer 88 and the tracking device 160. The method 510 proceeds to block 532 once line of sight has been reestablished. At block 532, the method either ends or returns to block 520 where the processor 102 continues to monitor for the presence of light of sight between the optical localizer 88 and the optical tracker 160.

In the method 510, however, a determination of whether a break condition is met may be determined in block 531. The break condition may be a number of repetitions and/or time to attempt to reestablish a line of site. The time may be if longer than 5 seconds has passed and/or more than 5 attempts to reestablish has been reached. It is understood, however, that any appropriate number may be provided and/or any appropriate break condition may be set. The break condition may be recalled at any appropriate time in the process 510. Thus, if the break condition is met then the process may go to the End block 532 rather than looping to block 528. If the break condition is not met, then the process 510 may loop to block 528.

In the method 510, the processor 102 in executing block 528 may determine a likely last location of the tracking device 160. This may be based on a last tracked location of the tracking device 160, known or last determined motion of the tracking device 160, etc. Thus, the likely position may be determined and used to searching for the tracking device 160 if line of site is not reestablished. Further, a search pattern may include a more random search, such as selected a distance from a last known location and searching in a selected pattern in the distance therefrom. For example, the rings noted above. This may allow for a search of an area, particularly, when a likely position does not achieve a reestablishment of the line of site. As noted above, however, a break condition can be met and the system can be reset and/or a manual line of site reestablishment may occur.

Also, the robotic arm 20 may move to achieve a reestablishment of a line of site. That is the servomotors may be activated to move the robotic arm 20 to move the tracking device 160 into a line of sight from the localizer 88. Thus, both the localizer 88 and the robotic arm 20 may be moved separately or in concert to achieve a reestablishment of a line of sight. It is understood that only one of the localizer 88 or the robotic arm 20 may move or both may move. Regardless, the process of moving may be similar for both localizer 88 and the robotic arm 20 to move.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors or processor modules, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A surgical navigation system comprising:
an optical tracking device;
an optical localizer configured to optically track the optical tracking device;
a mount assembly supporting the optical localizer; and
a processor configured to execute instructions to transmit commands to the mount assembly for actuating the mount assembly to position the optical localizer where there is line of sight between the optical localizer and the optical tracking device.

2. The surgical navigation system of claim 1, wherein the optical localizer is configured to generate or receive an optical signal within a navigation domain relative to a subject.

3. The surgical navigation system of claim 1, wherein the mount assembly includes:
a base;
an arm extending from the base;
a plurality of joints along the arm, each one of the plurality of joints including a servomotor configured to actuate the arm at the plurality of joints to move the optical localizer up, down, left, right, towards the optical tracking device, and away from the optical tracking device.

4. The surgical navigation system of claim 1, wherein the optical tracking device is mounted to a surgical instrument.

5. The surgical navigation system of claim 1, wherein the optical tracking device is mounted to a robotic arm.

6. The surgical navigation system of claim 5, wherein the robotic arm includes an end effector, the optical tracking device is mounted proximate to the end effector.

7. The surgical navigation system of claim 1, wherein the processor is configured to identify loss of line of sight between the optical localizer and the optical tracking device, and transmit commands to the mount assembly to reposition the optical localizer until line of sight between the optical localizer and the optical tracking device is reestablished.

8. The surgical navigation system of claim 1, wherein the processor is configured to:
identify location of the optical tracker in an instrument coordinate system;
identify location of the optical localizer in a localizer coordinate identify location of the optical localizer in the instrument coordinate system; and
system.

9. The surgical navigation system of claim 8, the processor further configured to, when line of sight between the optical localizer and the optical tracker is lost, transmit repositioning commands to the mount assembly for repositioning the optical localizer relative to a last known position of the optical localizer where line of sight was present between the optical localizer and the optical tracking device.

10. A surgical navigation system comprising:
an optical tracking device;
an optical localizer configured to optically track the optical tracking device;
a mount assembly including a base, an arm extending from the base to which the optical localizer is mounted, a plurality of joints along the arm, and a servomotor at each one of the plurality of joints configured to actuate the arm to move the optical localizer up, down, left, right, towards the optical tracking device, and away from the optical tracking device.
a processor configured to:
transmit commands to the mount assembly for actuating the servomotors to maneuver the arm and position the optical localizer where there is line of sight between the optical localizer and the optical tracking device;
identify location of the optical tracker in an instrument coordinate system;
identify location of the optical tracker in a localizer coordinate system;
identify location of the optical localizer in the instrument coordinate system; and
when line of sight between the optical localizer and the optical tracker is lost, transmit commands to the mount assembly for actuating the servomotor at one or more of the plurality of joints to move the arm and reposition the optical localizer relative to a last known position of the optical localizer where line of sight was present between the optical localizer and the optical tracking device.

11. The surgical navigation system of claim 10, wherein the optical localizer is configured to generate and receive an optical signal within a navigation domain relative to subject.

12. The surgical navigation system of claim 10, wherein the mount assembly is movable with six degrees of freedom.

13. The surgical navigation system of claim 10, wherein the optical tracking device is mounted to a surgical instrument.

14. The surgical navigation system of claim 10, wherein the optical tracking device is mounted to a robotic arm.

15. The surgical navigation system of claim 10, wherein the processor is configured to continue to transmit commands to the mount assembly for actuating the servomotor at one or more of the plurality of joints to move the arm and reposition the optical localizer until line of sight is reestablished between the optical localizer and the optical tracking device.

16. A method for maintaining line of sight between an optical localizer supported by an arm of a mount assembly and an optical tracking device of a surgical navigation system during a surgical procedure, the method comprising:
identifying. with a processor, a location of the optical tracker in an instrument coordinate system;
identifying, with the processor, the location of the optical tracker in a localizer coordinate system;
identifying, with the processor, the location of the optical localizer in the instrument coordinate system; and
when line of sight between the optical localizer and the optical tracker is lost, transmitting, by the processor, commands to the mount assembly for actuating the arm and repositioning the optical localizer relative to a last known position of the optical localizer where line of sight was present between the optical localizer and the optical tracking device.

17. The method of claim 16, wherein the optical localizer is configured to generate and receive an optical signal within a navigation domain relative to subject.

18. The method of claim 16, wherein the optical tracking device is mounted to a surgical instrument.

19. The method of claim 16, wherein the optical tracking device is mounted to a robotic arm.

20. The method of claim 16, wherein the processor is configured to continue to transmit commands to the mount assembly for actuating the servomotor at one or more of the plurality of joints to move the arm and reposition the optical localizer until line of sight is reestablished between the optical localizer and the optical tracking device.
